(19) United States
(12) Patent Application Publication
(10) Pub. No.: US 2024/0335479 A1
(43) Pub. Date: Oct. 10, 2024

(54) BIOLOGICAL MATERIAL IMPREGNATED WITH A SOLUTION COMPRISING EXOSOMES

(71) Applicant: TBF GENIE TISSULAIRE (TBF), Mions (FR)

(72) Inventor: Laurence BARNOUIN, Venerieu (FR)

(73) Assignee: TBF GENIE TISSULAIRE (TBF), Mions (FR)

(21) Appl. No.: 18/575,571

(22) PCT Filed: Jun. 29, 2022
(86) PCT No.: PCT/EP2022/067938

§ 371 Date: Feb. 16, 2024

(30) Foreign Application Priority Data
Jun. 30, 2021 (FR) FR2107114

(51) Int. Cl. A61K 35/51; A61K 35/28; A61K 35/50

(52) U.S. Cl. CPC A61K 35/51; A61K 35/28; A61K 35/50

(57) ABSTRACT

Biological materials impregnated with exosomes and uses thereof in therapeutic treatments. It also relates to a biological material impregnation kit including a lyophilized biological material and a solution having exosomes. Finally, it relates to a method for obtaining an impregnated biological material.

The present invention relates to the field of biological materials impregnated with exosomes and uses thereof in therapeutic treatments.

Exosomes are a form of extracellular vesicle with a diameter of between 30 and 100 nm. They are surrounded by a lipid bilayer and float at a density of 1.13-1.19 g/ml in sucrose gradients. Vesicles exhibiting the characteristics of exosomes have been isolated from various different body fluids, including semen, blood, saliva, breast milk, amniotic fluid, ascites fluid, cerebrospinal fluid, or bile. Over the past decade, much research has been done on exosomes as a mode of intercellular communication, since exosomes are secreted by various cell types, including stem cells. Some studies indicate that mesenchymal stem cells secrete exosomes in vitro and that these exosomes have therapeutic properties.

Exosomes contain numerous proteins or nucleic acids of interest, such as growth factors, cytokines, heat shock proteins (HSP), amino acids, nucleic acids (DNA, RNA), metabolites, enzymes, etc. They also comprise membrane proteins that can act as receptors/ligands in different cell signaling pathways.

In the publication by Bakhtyar et al., Stem Cell Research & Therapy (2018) 9: 193, it is described that exosomes derived from mesenchymal cells present in Wharton's jelly have beneficial effects on healing.

Many other therapeutic applications of vesicular biotherapy are notably listed in the publication Aubertin, médecine/science 2021; 37: 1146-57, which also describes the current challenges associated with the means of administering extracellular vesicles, particularly exosomes, the current methods consisting in the injection of the secretome of the cells of interest, but these techniques are limited by the extremely short half-life of extracellular vesides in the circulation.

Likewise, patent application US2020/397945 discloses a formulation comprising a biological material in hydrated form, particularly Wharton's jelly, and exosomes derived from mesenchymal stem cells, for administration in the treatment of damage to cardiac structures. That formulation is administered by catheter, injection, or via a prosthesis. In that application, since the biological material is simply juxtaposed with the exosomes, it does not allow for the capture and sustained release of the exosomes.

Patent application US2018/228848 discloses a biological composition comprising a mixture of non-cellular compounds, particularly exosomes, derived from placental tissues for therapeutic administration. However, the method according to that application does not make it possible to obtain a means of administering exosomes that were not present in the starting placental tissue. It also does not provide a means for the sustained-release administration of exosomes.

Patent application WO2020/231702 discloses a composition comprising differentiated cells, an adhesive material in hydrated form, and exosomes which can originate from mesenchymal cells. In that application, the exosomes are retained on the surface of the adhesive material and not impregnated within it. The composition must then be formulated with pharmaceutical and/or cosmetic excipients before application.

Patent application WO2017/140914 filed by the applicant disdoses a method for preparing an allograft material forming a virally inactivated, lyophilized, and sterile membrane derived from mammalian placental tissues. It does not disclose materials impregnated with a solution comprising exosomes or means of administering them.

There is therefore interest in the development of new, improved forms of administration for these exosomes. In addition, a need exists for new forms of sustained-release administration which make it possible to reduce the frequency of administration and/or to avoid concentrations that are too high in a short period of time.

The applicant has demonstrated that, in the presence of a hydrated biological material, contact with a solution comprising exosomes does not allow the impregnation of the biological material with said solution.

Consequently, no biological material of the prior art is impregnated with a solution comprising exosomes.

The applicant has succeeded in developing a method for impregnating a lyophilized biological material without degrading the biological material. Lyophilization makes it possible to eliminate the water bound up in the biological material, thereby enabling the absorption and impregnation of the solution comprising exosomes. In particular, lyophilization makes it possible to eliminate the water retained by the proteoglycans of the biological material and enables impregnation in this proteoglycan network.

In addition, the impregnation method developed by the applicant has the advantage of also allowing for the impregnation of decellularized or devitalized tissues. Due to the low porosity of devitalized tissues, the impregnation of these tissues poses quite a challenge, as is described by DUBUS M., et al. (“Antibacterial and immunomodulatory properties of acellular Wharton's jelly matrix.” Biomedicines, vol. 10,2 227, Jan. 21, 2022).

The biological material impregnated with a solution comprising exosomes according to the invention thus provides a form of administration for exosomes that can be used in therapy.

This impregnated biological material also presents unprecedented exosome release kinetics, making the device particularly attractive for therapeutic applications. To wit, the impregnated biological material developed by the applicant constitutes a reservoir of exosomes, allowing for a progressive and sustained release of said exosomes after administration.

The impregnated biological material according to the present invention also has the advantage of being able to be used directly in therapy—i.e., without the need for an additional formulation or preparation step.

In particular, the applicant has succeeded in performing impregnation with exosomes materials derived from a placenta and/or from umbilical cord, particularly from the amniotic membrane or from Wharton's jelly, which themselves possess properties that are of biological interest.

The chorioamniotic membrane, which separates the fetus from the mothers endometrium in mammals, includes the amniotic membrane—or amnion, and the chorionic membrane—or chorion. These two membranes are connected by a spongy tissue membrane, also called the spongy layer, which is composed inter alia of collagen and proteoglycans, the spongy tissue membrane comprising protein bridges which are attached on either side to the amnion on the one hand and to the chorion on the other hand.

The amniotic membrane is the innermost layer of the chorioamniotic membrane. Its role is to protect the fetus and maintain amniotic fluid around it. This amniotic membrane is a very thin, transparent tissue comprising a plurality of layers, namely an epithelial layer, a basement membrane, a compact layer, and a fibroblast layer.

Non-vascularized and non-innervated, the amniotic membrane is rich in collagen and various growth factors and has properties that are involved in the healing process.

The amniotic membrane, obtained from the placenta after childbirth, is a tissue that has been used for over a hundred years in the treatment of burns and wounds. In fact, Davies used fetal membranes on both burns and ulcerated tissues as early as 1910. Trelford and Trelford-Sauder report that, in 1935, authors published clinical applications of amniotic membrane in vaginoplasty, conjunctival reconstruction, the treatment of burns or wounds, and treatments relating to intra-abdominal adhesion. Trelford et al. also report that Douglas used amnion to treat extensive wounds in 1952. For the first time, it was indicated that the stromal layer of the amniotic membrane, comprising the compact layer and the fibroblast layer, allowed for greater adhesion of the graft and thus enhanced the effectiveness thereof. In 1972, the work of Trelford et al. confirmed this fact. Gindraux et al. report that, starting in 1972, and especially since its rediscovery in 1995, other authors have confirmed all of the clinical applications that had been presented previously and have also reported new indications, such as for the genitourinary tract, stomach, larynx, oral cavity, head, and neck, whether in clinical trials or case reports.

The amniotic membrane is particularly rich in growth factors such as EGF (Epidermal Growth Factor), TGF (Transforming Growth Factor) and KGF (Keratinocyte Growth Factor), as well as hyaluronic acid. These growth factors, hyaluronic acid, and collagen present in the amniotic membrane are particularly suited to promoting biological healing processes.

Wharton's jelly is a gelatinous connective tissue surrounding the vein and two arteries of the umbilical cord of mammals. Wharton's jelly is a substance that is particularly rich in constituent elements of the extracellular matrix of connective tissues, particularly glycosaminoglycans and proteoglycans; as well as collagen fibers (types I, III, IV, and V). Wharton's Jelly also comprises many growth factors such as fibroblast growth factors (FGF), insulin-like growth factor I (IGF-1), transforming growth factor (TGF), platelet-derived growth factors (PDGF), and epidermal growth factors (EGF).

These structural properties of Wharton's jelly can be utilized for their properties in aiding in the healing of lesions, including skin lesions or ocular surface lesions. Indeed, these constituent elements of Wharton's jelly participate in improving the biological processes of healing and reducing inflammation in a patient.

Biological materials from the umbilical cord or placenta can serve as ideal matrices for the impregnation of active ingredients which will then be released during the use of these tissues for different therapeutic treatments.

The present invention relates to a method for obtaining an impregnated biological material, comprising the steps of:
a) providing a lyophilized biological material and a solution comprising exosomes,
b) bringing the lyophilized biological material into contact with the solution comprising exosomes,
c) obtaining an impregnated biological material according to the invention.

In one embodiment, the lyophilized biological material is as defined below.

In one embodiment, the solution comprising exosomes is as defined below.

In one embodiment, the method comprises a preliminary step of lyophilizing a starting biological material.

In one embodiment, the method comprises a preliminary step of culturing the cells of interest followed by a step of collecting the exosomes in the culture medium of the cells of interest.

In one embodiment, the collection of exosomes in the culture medium of the cells of interest is performed by centrifugation.

In one embodiment, the contacting of step b) is carried out by depositing the solution comprising the exosomes onto the surface of the lyophilized biological material.

In one embodiment, the duration of contacting in step b) is at least 15 seconds.

In one embodiment, the duration of contacting in step b) is approximately 1 minute.

In one embodiment, the method further comprises a step of lyophilizing the impregnated biological material after step c).

In one embodiment, the method according to the invention further comprises a sterilization step after step c).

In one embodiment, the method according to the invention comprises a preliminary step of sterilizing the starting biological material and/or the lyophilized biological material before step a).

In one embodiment, the sterilization steps are performed by irradiation.

In one embodiment, the sterilization steps are performed by gamma irradiation by exposing the latter to gamma radiation at 25-32 kGy.

In one embodiment, the method according to the invention comprises a prior step of virally inactivating the starting biological material and/or the lyophilized biological material before step a).

In one embodiment, the lyophilized biological material of steps a) and/or b) is virally inactivated and/or sterile.

In one embodiment, the method according to the invention further comprises a devitalization step after step c).

In one embodiment, the method according to the invention further comprises a devitalization step before step a).

In one embodiment, the method according to the invention further comprises, before step a), a step of shaping the lyophilized biological material.

In one embodiment, the method according to the invention further comprises, after step c), a step of shaping the impregnated biological material.

In one embodiment, a step of shaping the impregnated biological material by molding is also included after step c).

In one embodiment, the method according to the invention is characterized in that the lyophilized biological material and/or the impregnated biological material is shaped into a parallelepiped, disc, cylinder, cone, or sphere.

In one embodiment, the method according to the invention is characterized in that the lyophilized biological material and/or the impregnated biological material is shaped into a parallelepiped whose length and width are between 0.2 cm and 10 cm and whose height is between 0.2 cm and 1.0 cm.

In one embodiment, the method according to the invention is characterized in that the lyophilized biological material and/or the impregnated biological material is shaped into a parallelepiped whose length and width are between 0.1 cm and 10 cm and whose height is between 0.1 cm and 1.0 cm.

In one embodiment, the method according to the invention is characterized in that the lyophilized biological material and/or the impregnated biological material is shaped into a disc whose diameter is between 0.2 cm and 10.0 cm.

In one embodiment, the method according to the invention is characterized in that the lyophilized biological material and/or the impregnated biological material is shaped into a cylinder whose diameter is between 0.2 cm and 10.0 cm and whose height is between 0.2 cm and 1.0 cm.

In one embodiment, the method according to the invention is characterized in that the lyophilized biological material and/or the impregnated biological material is shaped into a cylinder whose diameter is between 0.15 cm and 2.0 cm and whose height is from 0.5 cm to 3 cm.

In one embodiment, the method according to the invention is characterized in that the lyophilized biological material and/or the impregnated biological material is shaped into a cylinder whose diameter is between 0.1 cm and 10.0 cm and whose height is between 0.1 cm and 10.0 cm.

In one embodiment, the method according to the invention is characterized in that the lyophilized biological material and/or the impregnated biological material is shaped into a sphere whose diameter is between 0.2 cm and 1.0 cm.

In one embodiment, the method according to the invention is characterized in that the lyophilized biological material and/or the impregnated biological material is shaped into a sphere whose diameter is between 0.1 cm and 1.0 cm.

In one embodiment, the method according to the invention is characterized in that the lyophilized biological material and/or the impregnated biological material is shaped into a cone whose plane diameter is between 0.2 cm and 1.0 cm and whose height is between 0.2 cm and 1.0 cm.

In one embodiment, the method according to the invention is characterized in that the lyophilized biological material and/or the impregnated biological material is in powder form.

In one embodiment, the method according to the invention further comprises, after step c), a step of preparing the impregnated biological material in a form suitable for parenteral administration.

In one embodiment, the method according to the invention further comprises, after step c), a step of preparing the impregnated biological material in a form suitable for application to the surface of the skin and/or to a mucous membrane and/or to the eyelash and/or in an anal fistula.

In one embodiment, the method according to the invention further comprises, after step c), a step of preparing the impregnated biological material in a form suitable for application in the vitreous body of the eye or as a subconjunctival injection.

In one embodiment, the method according to the invention further comprises, after step c), a step of preparing the impregnated biological material in a form suitable for administration in the form of an implant.

For the purposes of the present invention, the term “biological starting material” is understood to mean any material derived and/or isolated from human, animal, or plant tissues. By convention, “biological starting material” will refer to a material that is neither lyophilized nor impregnated.

In one embodiment, the starting biological material comprises proteoglycans.

In one embodiment, the starting biological material is a connective tissue comprising proteoglycans.

In one embodiment, the starting biological material is a connective tissue rich in proteoglycans.

In one embodiment, the starting biological material has the consistency of a solid or gel.

In one embodiment, the starting biological material is not a liquid.

In one embodiment, the cells of the starting biological material are devitalized.

For the purposes of the present invention, the term “devitalized cell” is understood to mean a cell whose continuity of cellular and/or nuclear membranes is altered by a physical and/or chemical process but whose cellular content, particularly DNA or RNA, is not eliminated.

In one embodiment, the devitalization of the cells is carried out by means of at least one freezing/thawing cycle.

In one embodiment, the starting biological material is decellularized.

The size of the starting biological material according to the invention will be selected appropriately by those skilled in the art, particularly if the starting biological material is to be used therapeutically, so that it has a size that is suited to the region that is to be treated.

In one embodiment, the starting biological material is characterized in that the starting biological material originates from one or more tissues of human origin.

In one embodiment, the starting biological material is selected from the list consisting of the amniotic membrane or a starting biological material from human placenta or human umbilical cord, particularly human Wharton's jelly and/or human amniotic membrane.

In one embodiment, the starting biological material is characterized in that it originates from placenta or umbilical cord.

In one embodiment, the starting biological material comprises umbilical cord wall.

In one embodiment, the starting biological material is Wharton's jelly.

“Wharton's jelly” is understood to refer to the gelatinous biological tissue present in the umbilical cord of mammals from which the vein and the two arteries that are naturally included within the gelatinous biological tissue have been removed. In the present invention, the term “Wharton's jelly” can be understood as including or not including the amniotic membrane surrounding the Wharton's jelly of the umbilical cord.

In one embodiment, the term “Wharton's jelly” is understood to not include thick collagen fibers and/or lacunae and vascular walls (villi and intervillous spaces).

In one embodiment, the starting biological material in the form of Wharton's jelly is characterized in that it is obtained according to the method described in WO2019/038411.

In one embodiment, Wharton's jelly is obtained according to a method comprising the steps of:
a) providing a segment of Wharton's jelly from which the vein and arteries have been removed;
b) a viral inactivation treatment of the Wharton's jelly segment is performed in order to obtain a virally inactivated Wharton's jelly segment;
c) the segment of virally inactivated Wharton's jelly is ground in order to obtain a homogeneous grind of virally inactivated Wharton's jelly.

In one embodiment, the Wharton's jelly does not comprise umbilical vessels.

In one embodiment, the starting biological material comprises Wharton's jelly and the amniotic membrane surrounding the same.

In one embodiment, the starting biological material is an amniotic membrane and/or originates from an amniotic membrane.

“Amniotic membrane” is understood to refer to the envelope of tissue which develops around the embryo and then the fetus in mammals during pregnancy. Its role is to protect the developing organism by maintaining amniotic fluid around it. It attaches to the second membrane, which is the chorion. The amniotic membrane includes the following physiological sublayers: the epithelial cell layer, the basement membrane, the compact layer, the fibroblast layer, and the spongy layer.

In one embodiment, the starting biological material comprises the spongy layer of the amniotic membrane.

The spongy layer of the amniotic membrane has the advantage of being very rich in proteoglycans.

In one embodiment, the starting biological material in the form of amniotic membrane is characterized in that it is obtained according to the method described in WO2017/140914.

In one embodiment, the starting biological material is sterile.

In one embodiment, the starting biological material is virally inactivated.

“Viral inactivation” is understood to refer to a technique which makes it possible to substantially or completely, and definitively, reduce the ability of viruses to act. Defined as inactivated, these lose their pathogenic and replicative capacities through a decrease in their population of 4 log during residual titrations which follow one or two independent chemical steps, whether on enveloped or non-enveloped viruses, DNA, or RNA.

Such a method is described, for example, in documents WO2017/140914 or WO2019/038411 under the name of TBF GENIE TISSULAIRE.

In one embodiment, the device according to the invention is characterized in that the starting biological material is virally inactivated according to the two chemical viral inactivation steps of the method described in WO2017/140914 or WO2019/038411.

In one embodiment, the two steps are a chemical treatment step with an alcohol that is particularly effective against enveloped viruses and a chemical treatment step with a peroxide that is particularly effective against naked viruses.

In one embodiment, the first virus-inactivating chemical treatment step is the application of a wash, or the soaking of the latter in a bath, composed of a first virus-inactivating agent, namely ethanol. Washing with purified water or soaking in a bath of purified water can be advantageously carried out after this step.

According to one embodiment, the first viral inactivation agent is ethanol with an alcohol content of between 50% and 80%, preferably 70% v/v.

According to another embodiment, the first viral inactivation step is performed through treatment with 70% v/v ethanol for approximately 60 minutes.

The second step of the virus-inactivating chemical treatment is the application of a wash, or the soaking of the latter in a bath, composed of a second virus-inactivating agent, namely hydrogen peroxide.

As stated above, it is known that treatment with hydrogen peroxide solutions is only effective on non-enveloped viruses at concentrations of greater than 10%.

The second viral inactivation agent is hydrogen peroxide in a form selected from among an aqueous solution and a gas.

According to one embodiment, the second viral inactivation agent is hydrogen peroxide in the form of an aqueous solution in a concentration of between 3% and 30% w/v.

In one embodiment, the starting biological material is sterile.

For the purposes of the present invention, the term “sterile” is understood to mean a material that is devoid of germs either naturally or because it has been sterilized.

Sterilization can be carried out by any method conventionally known to those skilled in the art.

In one embodiment, the sterilization is performed by gamma irradiation.

In one embodiment, the lyophilized biological material is sterilized.

For the purposes of the present invention, the expression “lyophilized biological material” is understood to mean a starting biological material that has undergone at least one lyophilization step and has not been rehydrated or impregnated.

In one embodiment, the lyophilized biological material is sterile and/or virally inactivated.

In one embodiment, the cells of the lyophilized biological material are devitalized.

In one embodiment, the lyophilized biological material is decellularized.

For the purposes of the present invention, “lyophilization” is understood to refer to a technique aimed at drying a previously frozen product by sublimation. More precisely, the liquid to be removed from the product is first transformed into ice by freezing; then the ice is sublimated by primary desiccation under a vacuum; finally, through secondary desiccation, the water molecules on the surface of the product are extracted by desorption.

In one embodiment, lyophilization is carried out under the following conditions:
freezing in two steps:
   the first freezing step takes place at an acclimation temperature that is selected so as not to damage the structural, functional, and biological integrity of the biological material;
   the second freezing step takes place at the final freezing temperature, which is lower than the acclimation temperature;
lyophilization in two main steps, termed primary and secondary:
   the primary lyophilization step is carried out through application of a vacuum of approximately 200 microbars and an ascending temperature profile;
   the secondary lyophilization step is carried out through application of a vacuum of approximately 50 microbars and a descending temperature profile.

In one embodiment, the acclimation temperature is between −5 and −20° C., and the final freezing temperature is between −40 and −60° C.

The ascending temperature profile is advantageously a profile according to which the lyophilization temperature is initially set at a low initial temperature and then raised toward a final primary lyophilization temperature in one or more intermediate ascending temperature steps. The descending temperature profile is advantageously a profile according to which the lyophilization temperature is initially set at a temperature higher than the final temperature of the primary lyophilization step and then lowered to a final secondary lyophilization temperature which is higher than the initial temperature of the primary lyophilization step.

In one embodiment, the lyophilized biological material is virally inactivated and/or sterile.

Viral inactivation treatments as well as lyophilization destroy the membranes of the exosomes that would be naturally present in the tissues serving as matrices, so the environment of these matrices after these treatments is suitable for impregnation with solutions comprising exosomes, it being possible for these exosomes to originate from many different cell types.

The content of the exosomes according to the present invention depends on the type of cells from which they were isolated.

It is possible for the starting biological material to include exosomes before impregnation. Therefore, as a matter of convention, reference will be made to “exosomes according to the invention” to designate exosomes which were not present in the starting biological material and/or the lyophilized biological material before impregnation with the solution comprising exosomes according to the invention.

The exosomes according to the present invention can be isolated from different cell types of interest depending on the pathology to be treated. The isolation can be achieved by any technique known to those skilled in the art.

In one embodiment, the exosomes according to the present invention are isolated by centrifugation, filtration, ultrafiltration, and/or immunoprecipitation of the culture medium of the cells of interest.

The exosomes according to the present invention can originate from a single cell type or from a mixture of different cell types.

In one embodiment, the cells of interest are selected from the group consisting of macrophages, blood platelets, dendritic cells, mesenchymal stem cells, induced pluripotent stem cells, bone marrow cells, adipose tissue, and/or umbilical cord tissue and/or are purified from biological fluids.

In one embodiment, the cells of interest are genetically modified cells.

In one embodiment, the exosomes according to the invention originate from the treated patient (autologous) or from one or more donors (allogeneic).

In one embodiment, the exosomes according to the invention are derived from mesenchymal stem cells.

In one embodiment, the exosomes according to the invention are derived from human mesenchymal stem cells.

In one embodiment, the human mesenchymal stem cells according to the invention are obtained using a method that does not require the destruction of the embryo.

In one embodiment, the mesenchymal stem cells according to the invention originate from umbilical cord.

In one embodiment, the mesenchymal stem cells according to the invention are derived from human umbilical cord.

For the purposes of the present invention, “the solution comprising exosomes according to the invention” or “the solution according to the invention” are used interchangeably.

In one embodiment, the solution comprising exosomes according to the invention comprises a therapeutically effective amount of exosomes according to the invention.

For the purposes of the present invention, the term “therapeutically effective quantity of exosomes” is understood to mean the quantity of exosomes according to the invention which eliminates, attenuates, or relieves the symptoms for which it is administered.

In one embodiment, the solution comprising exosomes according to the invention comprises a quantity of at least 10⁶ exosomes according to the invention.

In one embodiment, the solution comprising exosomes according to the invention comprises a quantity of at least 10⁹ exosomes according to the invention.

In one embodiment, the solution comprising exosomes according to the invention comprises a quantity of at least 10¹¹ exosomes according to the invention.

In one embodiment, the solution comprising exosomes according to the invention is an aqueous solution.

In one embodiment, the solution according to the invention further comprises the culture medium of the cells of interest from which the exosomes have been extracted.

In one embodiment, the solution according to the invention further comprises saline phosphate buffer.

In one embodiment, the solution according to the invention further comprises another active ingredient.

For the purposes of the present invention, “impregnation” and/or “impregnated” is understood to mean that the solution comprising exosomes penetrates into the lyophilized biological material and spreads therein, diffuses therein.

The impregnation therefore consists of introducing the solution comprising the exosomes according to the invention into the lyophilized biological material according to the invention. The lyophilized biological material is therefore at least partially rehydrated and the exosomes according to the invention are embedded in the proteoglycan network of the impregnated biological material according to the invention, enabling it to be lyophilized again in order to be preserved without losing its content of exosomes according to the invention.

The impregnation according to the invention thus makes it possible to add a defined quantity of exosomes according to the invention that was not present in the biological material before impregnation.

The quantification of the impregnation can be carried out by any method conventionally known to those skilled in the art.

In one embodiment, the quantification of the impregnation is carried out by means of an ELISA immunoenzymatic method for detecting exosomes in the impregnation medium using a biological material before impregnation according to the invention as a negative control.

In one embodiment, this involves impregnation with at least 10⁶ exosomes according to the invention.

In one embodiment, this involves impregnation with at least 10⁹ exosomes according to the invention.

In one embodiment, this involves impregnation with at least 10¹¹ exosomes according to the invention.

In one embodiment, this involves impregnation with at least 90% of the exosomes present in the solution comprising exosomes according to the invention.

In one embodiment, this involves impregnation with at least 95% of the exosomes present in the solution comprising exosomes according to the invention.

In one embodiment, this involves impregnation with at least 98% of the exosomes present in the solution comprising exosomes according to the invention.

The present invention also relates to a biological material impregnated with a solution comprising exosomes.

In one embodiment, the impregnated biological material originates from the impregnation of a lyophilized biological material with a solution comprising exosomes according to the invention.

In one embodiment, the impregnated biological material according to the invention has the consistency of a solid or gel.

In one embodiment, the impregnated biological material according to the invention is not a liquid.

In one embodiment, the impregnated biological material comprises proteoglycans.

In one embodiment, the impregnated biological material is a connective tissue comprising proteoglycans.

In one embodiment, the impregnated biological material is a connective tissue rich in proteoglycans.

In one embodiment, the impregnated biological material according to the invention is in a form that is suitable for parenteral administration.

In one embodiment, the impregnated biological material according to the invention has a shape that is suitable for in situ administration.

In one embodiment, the impregnated biological material according to the invention has a shape that is suitable for peritoneal administration.

In one embodiment, the impregnated biological material according to the invention has a shape that is suitable for administration in the form of an implant.

In one embodiment, the impregnated biological material according to the invention has a shape that is suitable for application to the surface of the skin and/or to a mucous membrane and/or to the eyelash and/or in an anal fistula.

In one embodiment, the impregnated biological material according to the invention comprises a therapeutically effective quantity of exosomes according to the invention.

In one embodiment, the impregnated biological material is impregnated with a quantity of 10⁶ exosomes according to the invention.

In one embodiment, the impregnated biological material is impregnated with a quantity of 10⁹ exosomes according to the invention.

In one embodiment, the impregnated biological material is impregnated with a quantity of 10¹¹ exosomes according to the invention.

In one embodiment, the impregnated biological material has the shape of a parallelepiped, disc, cylinder, cone, or sphere or is a powder.

In one embodiment, the impregnated biological material has the shape of a parallelepiped whose length and width are between 0.1 cm and 10 cm and whose height is between 0.1 cm and 1.0 cm.

In one embodiment, the impregnated biological material has the shape of a parallelepiped whose length and width are between 0.2 cm and 10 cm and whose height is between 0.2 cm and 1.0 cm.

In one embodiment, the impregnated biological material has the shape of a disc whose diameter is between 0.2 cm and 10.0 cm.

In one embodiment, the impregnated biological material has a cylindrical shape whose diameter is between 0.1 cm and 10.0 cm and whose height is between 0.1 cm and 10.0 cm.

In one embodiment, the impregnated biological material has a cylindrical shape whose diameter is between 0.2 cm and 10.0 cm and whose height is between 0.2 cm and 1.0 cm.

In one embodiment, the impregnated biological material has the shape of a cylinder whose diameter is between 0.15 cm and 2.0 cm and whose height is between 0.5 cm and 3 cm.

In one embodiment, the impregnated biological material has the shape of a sphere whose diameter is between 0.1 cm and 1.0 cm.

In one embodiment, the impregnated biological material has the shape of a sphere whose diameter is between 0.2 cm and 1.0 cm.

In one embodiment, the impregnated biological material has the shape of a cone whose plane diameter is between 0.2 cm and 1.0 cm and whose height is between 0.2 cm and 1.0 cm.

In one embodiment, the biological material is impregnated with at least a second active ingredient.

In one embodiment, the impregnated biological material according to the invention is characterized in that the at least one second active ingredient is selected from the group consisting of antibiotics, antiseptics, antivirals, monoclonal antibodies, semi-synthetic metalloprotease inhibitors, immunosuppressive agents, anti-inflammatories, antifungals, antiallergics, anesthetics, or immunoadhesive proteins, or agents for preventing dry eyes, alone or in combination.

The at least second active ingredient may be an active ingredient which is or is not naturally present in the starting biological material.

In one embodiment, the impregnated biological material is characterized in that the at least one second active ingredient is selected from the group consisting of antibiotics. Some examples of usable antibiotics are given hereafter tetracyclines (daunomycin, tetracycline, chloretetracydine, oxytetracycline, etc.), glycopeptides (vancomycin, etc.), aminoglycosides (gentamycin, etc.), aminosides (tobramycin, neomycin, etc.), fluoroquinolones (ciprofloxacin, moxifloxacin, etc.), quinolones (gatifloxacin, etc.), polypeptides (bacitracin, polymyxin, etc.), phenicols (chloramphenicol, etc.), macrolides (erythromycin, etc.), sulfonamides (sulfacetamide, sulfamethoxazole, sulfisoxazole, etc.), cephalosporins (cefradoxil, cefoxitin, etc.), and any other antibiotic.

In one embodiment, the impregnated biological material is characterized in that the at least one second active ingredient is selected from the group consisting of steroidal anti-inflammatories (SAIDs) and/or non-steroidal anti-inflammatory drugs (NSAIDs). Some examples of SAIDs are given hereafter triamcinolone, dexamethasone, prednisolone, hydrocortisone, corticosterone, fluocinolone, prednisolone, methylprednisolone, fluorometholone, betamethasone, tetrahydrocortisol, rimexolone, etc. Some examples of NSAIDs are given hereafter indomethacin, nepafenac, didofenac, bromfenac, ketorolac, suprofen, etc.

In one embodiment, the impregnated biological material is characterized in that the at least one second active ingredient is selected from the group consisting of immunosuppressants, with a non-limitative list being given hereafter: dexamethasone, betamethasone, etc.

In one embodiment, the impregnated biological material is characterized in that the at least one second active ingredient is selected from the group consisting of antivirals, with a non-limitative list being given hereafter: gancidovir, trifluorothymidine, acyclovir, DDI, AZT, foscamet, vidarabine, trifluridine, idoxuridine, ribavirin, protease inhibitors, anti-cytomegalovirus agent, etc.

In one embodiment, the impregnated biological material is characterized in that the at least one second active ingredient is selected from the group consisting of antifungals, with a non-limitative list being given hereafter: fluconazole, nitrofurazone, amphotericin B, ketoconazole, etc.

In one embodiment, the impregnated biological material is characterized in that the at least one second active ingredient is selected from the group consisting of antiallergics, with a non-limitative list being given hereafter: methapyrilene, chlorpheniramine, pyrilamine, prophenpyridamine, etc.

In one embodiment, the impregnated biological material is characterized in that the at least one second active ingredient is selected from the group consisting of anesthetics, with a non-limitative list being given hereafter: lidocaine, mepivacaine, etc.

In one embodiment, the impregnated biological material is characterized in that the at least one second active ingredient is selected from the group consisting of agents which enable prevention of dry eyes, with a non-limitative list being given hereafter azithromycin, cyclosporin, lubricants, etc.

In one embodiment, the cells of the impregnated biological material are devitalized.

In one embodiment, the impregnated biological material is decellularized.

In one embodiment, the impregnated biological material according to the invention is sterile and/or virally inactivated.

In one embodiment, the impregnated biological material according to the invention is lyophilized.

A biological material impregnation kit comprising at least two independent means:
a) a lyophilized biological material,
b) a solution comprising exosomes.

In one embodiment, the impregnation kit according to the invention comprises a lyophilized biological material, as described above.

In one embodiment, the impregnation kit according to the invention comprises a solution comprising exosomes according to the invention, as described above.

Another object of the present invention relates to a device for administering exosomes comprising the biological material impregnated with a solution comprising exosomes according to the invention.

In one embodiment, the administration device according to the invention consists of a biological material impregnated with a solution comprising exosomes according to the invention.

In one embodiment, the administration device according to the invention is a device for the sustained-release delivery of exosomes.

In one embodiment, the device according to the invention allows for the release of exosomes for at least 4 hours from administration.

In one embodiment, the device according to the invention allows for the release of exosomes for at least 24 hours from administration.

In one embodiment, the device according to the invention allows for the release of exosomes for a period of from 24 to 72 hours from administration.

In one embodiment, the device according to the invention allows for the release of exosomes for at least 72 hours from administration.

In one embodiment, the device according to the invention allows for the release of exosomes for a period of from 4 hours to 1 week from administration.

In one embodiment, the device according to the present invention is applied to the surface of the skin and/or to a mucous membrane and/or to the eyelash and/or in an anal fistula.

In one embodiment, the device according to the invention is an implantable device.

In one embodiment, the device according to the invention is an eye dropper.

Another object of the present invention relates to a biological material impregnated with a solution comprising exosomes according to the invention for therapeutic use.

One embodiment of the present invention also relates to the biological material impregnated with a solution comprising exosomes according to the invention for therapeutic use in regenerative medicine and/or for the treatment and/or prevention of Crohn's disease and/or or fistulas and/or chronic bowel disease and/or graft-versus-host disease and/or intestinal inflammation and/or stroke and/or osteoarthritis and/or or respiratory distress syndrome and/or burns and/or cardiac myopathies and/or esophageal stenoses and/or chronic heart failure and/or cancer, particularly of the colon and/or breast and/or lung and/or pancreas and/or melanomas, and/or lysomal overload diseases, particularly Gaucher disease and/or Fabry disease, and/or mucopolysaccharidosis type III and/or Sanfilippo syndrome and/or bronchopulmonary dysplasia and/or chronic renal failure and/or mucositis after chemo- or radiotherapy treatment, and/or type I diabetes and/or gastroduodenal ulcers and/or pneumonia and/or venous ulcers and/or acute respiratory distress syndrome and/or Alzheimer's-related dementia and/or acute myocardial infarction and/or chronic post-surgical inflammation of the temporal bone and/or periodontitis and/or dry eye and/or macular degeneration and/or neuralgia and/or depression and/or dementia and/or dystrophic epidermolysis bullosa.

Another object of the present invention relates to a method of therapeutic treatment comprising a step of administering the biological material impregnated with a solution comprising exosomes according to the invention.

Another object of the present invention relates to a method of therapeutic treatment in regenerative medicine and/or for the treatment and/or prevention of Crohn's disease and/or or fistulas and/or chronic bowel disease and/or graft-versus-host disease and/or intestinal inflammation and/or stroke and/or osteoarthritis and/or or respiratory distress syndrome and/or burns and/or cardiac myopathies and/or esophageal stenoses and/or chronic heart failure and/or cancer, particularly of the colon and/or breast and/or lung and/or pancreas and/or melanomas, and/or lysomal overload diseases, particularly Gaucher disease and/or Fabry disease, and/or mucopolysaccharidosis type III and/or Sanfilippo syndrome and/or bronchopulmonary dysplasia and/or chronic renal failure and/or mucositis after chemo- or radiotherapy treatment, and/or type I diabetes and/or gastroduodenal ulcers and/or pneumonia and/or venous ulcers and/or acute respiratory distress syndrome and/or Alzheimer's-related dementia and/or acute myocardial infarction and/or chronic post-surgical inflammation of the temporal bone and/or periodontitis and/or dry eye and/or macular degeneration and/or neuralgia and/or depression and/or dementia and/or dystrophic epidermolysis bullosa comprising a step of administering the biological material impregnated with a solution comprising exosomes according to the invention.

Another object of the present invention relates to a device for administering exosomes comprising the biological material impregnated with a solution comprising exosomes according to the invention for therapeutic use.

One embodiment of the present invention also relates to the device for administering exosomes comprising the biological material impregnated with a solution comprising exosomes according to the invention for therapeutic use in regenerative medicine and/or for the treatment and/or prevention of Crohn's disease and/or or fistulas and/or chronic bowel disease and/or graft-versus-host disease and/or intestinal inflammation and/or stroke and/or osteoarthritis and/or or respiratory distress syndrome and/or burns and/or cardiac myopathies and/or esophageal stenoses and/or chronic heart failure and/or cancer, particularly of the colon and/or breast and/or lung and/or pancreas and/or melanomas, and/or lysomal overload diseases, particularly Gaucher disease and/or Fabry disease, and/or mucopolysaccharidosis type III and/or Sanfilippo syndrome and/or bronchopulmonary dysplasia and/or chronic renal failure and/or mucositis after chemo- or radiotherapy treatment, and/or type I diabetes and/or gastroduodenal ulcers and/or pneumonia and/or venous ulcers and/or acute respiratory distress syndrome and/or Alzheimer's-related dementia and/or acute myocardial infarction and/or chronic post-surgical inflammation of the temporal bone and/or periodontitis and/or dry eye and/or macular degeneration and/or neuralgia and/or depression and/or dementia and/or dystrophic epidermolysis bullosa.

Another object of the present invention relates to a method of therapeutic treatment comprising a step of administering the device for administering exosomes comprising the biological material impregnated with a solution comprising exosomes according to the invention.

Another object of the present invention relates to a method of therapeutic treatment in regenerative medicine and/or for the treatment and/or prevention of Crohn's disease and/or or fistulas and/or chronic bowel disease and/or graft-versus-host disease and/or intestinal inflammation and/or stroke and/or osteoarthritis and/or or respiratory distress syndrome and/or burns and/or cardiac myopathies and/or esophageal stenoses and/or chronic heart failure and/or cancer, particularly of the colon and/or breast and/or lung and/or pancreas and/or melanomas, and/or lysomal overload diseases, particularly Gaucher disease and/or Fabry disease, and/or mucopolysaccharidosis type III and/or Sanfilippo syndrome and/or bronchopulmonary dysplasia and/or chronic renal failure and/or mucositis after chemo- or radiotherapy treatment, and/or type I diabetes and/or gastroduodenal ulcers and/or pneumonia and/or venous ulcers and/or acute respiratory distress syndrome and/or Alzheimer's-related dementia and/or acute myocardial infarction and/or chronic post-surgical inflammation of the temporal bone and/or periodontitis and/or dry eye and/or macular degeneration and/or neuralgia and/or depression and/or dementia and/or dystrophic epidermolysis bullosa comprising a step of administering the device for administering exosomes comprising the biological material impregnated with a solution comprising exosomes according to the invention.

### EXAMPLES

### Example 1: Preparation of a Lyophilized Biological Material According to the Invention Consisting of Amniotic Membrane

A properly informed and consenting donor in accordance with the requirements of the Declaration of Helsinki offers the placental tissue resulting from childbirth as a donation. Due to health requirements relating to donations of tissues and cells of human origin, prior qualification of the donor is systematic. This qualification involves screening for HIV, hepatitis B, C, HTLV viruses, and the Treponema pallidum bacterium responsible for syphilis.

Placental tissue is collected as soon as possible in the delivery room following childbirth. It can be advantageously placed in a sterile box then frozen at a temperature of −20° C.

The following procedure is carried out in a sterile room in the laboratory:

The amniotic membrane with the spongy layer is isolated from the placenta, and the chorion is removed and cleaned.

This insulated fabric is stored dry at a temperature of −20° C. or −80° C. for up to two years or treated immediately.

The amniotic membrane undergoes a succession of baths which ensure the chemical treatment thereof. This treatment aims to disinfect and, in particular, to virally inactivate the amniotic membrane. The liquid medium is stirred gently at approximately 30 rotations per minute (rpm) during each bath to ensure homogeneous penetration of the solvents into the tissues.

First, the amniotic membrane is placed in a bath of purified water at room temperature for approximately 3 hours. This step ensures both the thawing of the physiological tissue and a first step of cell lysis by osmotic pressure.

The amniotic membrane is then transferred to a decontaminating bath composed of 70% v/v ethanol by volume of ethanol relative to the total volume of the solution at room temperature for approximately 1 hour.

Washing is performed in purified water for approximately 15 minutes at room temperature in order to remove the ethanol.

In order to ensure the second step of decontaminating treatment, the amniotic membrane is transferred to a bath composed of hydrogen peroxide at 30% w/v by weight of hydrogen peroxide relative to the total volume of the solution at room temperature for about 15 minutes.

Then the amniotic membrane is transferred to a decontaminating bath composed of hydrogen peroxide at 3% w/v by weight of hydrogen peroxide relative to the total volume of the solution at room temperature for approximately 1 hour.

The chemical action applied to the amniotic membrane can then be neutralized in two baths containing sodium hydroxide diluted to a pH of 8.5. The neutralization baths are carried out at room temperature for approximately 15 minutes.

In order to ensure a rebalancing of the pH and best eliminate the organic residues detaching from the tissue of interest, the amniotic membrane is transferred to two saline phosphate buffer baths in order to ensure the physiological rebalancing thereof. The baths are carried out at room temperature for approximately 15 minutes.

Finally, the amniotic membrane is transferred to a final bath in purified water, at room temperature, for at least 15 minutes and up to approximately 1 hour.

An amniotic membrane with a disinfected and virally inactivated spongy layer is obtained.

Following this stage relating to chemical treatments, the amniotic membrane undergoes a lyophilization treatment.

On a stainless steel tray, the amniotic membrane is placed between two layers of mesh methylcellulose support filters in order to facilitate water vapor exchange.

The assembly described above is transferred to a lyophilizer, where a freezing step followed by a lyophilization step are carried out according to the following specifications:

### Freezing:

The first freezing step is carried out at a temperature of −10° C. for 5 minutes, then at −15° C. for 90 minutes;
the second freezing step is carried out at a temperature of −50° C. for 125 minutes;

### Lyophilization:

A primary lyophilization step is carried out through application of a vacuum of 200 microbars and a temperature of +10° C. for 8 hours, followed by a temperature of +25° C. for 150 minutes;
a secondary lyophilization step is carried out through application of a vacuum of 50 microbars and a temperature of +35° C. for 5 hours, followed by a temperature of +25° C. for 1 hour.

A final sterilization step is carried out by exposing the amniotic membrane to gamma radiation at 25-32 kGrays.

A virally inactivated, lyophilized, and sterilized biological material consisting of an amniotic membrane with a spongy layer is obtained.

### Example 2: Preparation of a Lyophilized Biological Material According to the Invention Consisting of a Disc of Wharton's Jelly

A properly informed and consenting donor in accordance with the requirements of the Declaration of Helsinki offers the umbilical cord resulting from childbirth as a donation. Due to health requirements relating to donations of tissues and cells of human origin, prior qualification of the donor is systematic. This qualification involves screening for HIV, hepatitis B, C, HTLV viruses, and the Treponema pallidum bacterium responsible for syphilis.

The umbilical cord is retrieved as soon as possible in the delivery room. It is advantageously placed in a sterile box comprising an NaCl solution at 4° C.

The following procedure is carried out in a sterile room in the laboratory:

A segment of 20 cm to 50 cm in length is isolated per section of the umbilical cord.

The umbilical cord is rinsed and hydrated in successive purified water baths, under gentle stirring, for 4 hours.

The blood vessels of the umbilical cord segment are identified and separated from the rest of the segment in order to retain only Wharton's jelly and the amniotic membrane surrounding the same. The Wharton's jelly and the amniotic membrane are used in the rest of the process under the general name of Wharton's jelly, because the quantity by weight of membrane is negligible compared to the quantity by weight of Wharton's jelly.

The Wharton's jelly is lyophilized at a temperature of −20° C. or −80° C.

During chemical treatment, the Wharton's jelly is thawed in the open air, at room temperature, for 5 minutes. This freezing step, followed by thawing, ensures the substantial devitalization of the biological material.

The Wharton's jelly undergoes a succession of baths which ensure the chemical treatment thereof. This treatment aims to disinfect and, in particular, to virally inactivate the Wharton's jelly. Gentle linear stirring of the liquid medium is applied during each bath to ensure homogeneous penetration of the solvents into the tissues.

First, the Wharton's jelly is placed in a bath of purified water at room temperature for approximately 3 hours. This step ensures both the end of the thawing of the physiological tissue and a step of cell lysis by osmotic pressure.

The Wharton's jelly is then transferred to a decontaminating bath composed of 70% v/v ethanol at room temperature for approximately 1 hour.

Washing is performed in purified water for approximately 15 minutes at room temperature in order to remove the ethanol.

To ensure the second step of decontamination treatment, the Wharton's jelly is transferred to a bath composed of 30% w/v hydrogen peroxide at room temperature for approximately 15 minutes.

The Wharton's jelly is then transferred to a decontaminating bath composed of 3% w/v hydrogen peroxide at room temperature for approximately 1 hour. The Wharton's jelly obtained is virally inactivated. The chemical action applied to the virally inactivated Wharton's jelly is then neutralized in a bath containing sodium hydroxide diluted to a pH of around 8.5. The treatment with the neutralization bath is carried out at room temperature for approximately 15 minutes.

In order to ensure a rebalancing of the pH and best eliminate organic residues detaching from the tissue of interest, the virally inactivated Wharton's jelly is transferred to a bath of physiological buffer (PBS) in order to ensure the physiological rebalancing thereof. The bath is carried out at room temperature for approximately 15 minutes.

Finally, the Wharton's jelly is transferred to two successive baths with purified water, at room temperature, for at least 15 minutes and up to approximately 1 hour.

At this stage, we can consider that the Wharton's jelly obtained according to this chemical treatment is a largely disinfected, particularly virally inactivated, Wharton's jelly.

Following this stage relating to chemical treatments, the virally inactivated Wharton's jelly undergoes a grinding step.

The Wharton's jelly is inserted into a Retsch MM400 vibrating ball mill equipped with a 35 ml zirconium oxide bowl. The Wharton's jelly takes up about ⅓ of the bowl. A zirconium oxide ball with a diameter of 20 mm is added to the bowl with the Wharton's jelly. Grinding is performed at a frequency of 3 Hz for 1 minute. The ball with a diameter of 20 mm is collected, and 9 zirconium oxide balls with a diameter of 10 mm are added to the bowl. A second grinding is performed at a frequency of 30 Hz for 3 minutes. A third grinding is performed with sixty 5 mm balls for 3 minutes at a frequency of 30 Hz.

The substance obtained is a homogeneous gelled liquid substance.

The substance obtained is then placed into a stainless steel mold. This is achieved using a 2.5 ml syringe.

The substance obtained is then in the form of a disc.

Following this stage relating to grinding and shaping, the virally inactivated and ground Wharton's jelly undergoes lyophilization.

The virally inactivated and ground Wharton's jelly is placed on a stainless steel tray.

This is all transferred to a lyophilizer, where a freezing step followed by a lyophilization step are carried out according to the following specifications:

### Freezing:

The first freezing step is carried out at an acclimation temperature that is selected so as not to damage the structural, functional, and biological integrity of the virally inactivated and ground Wharton's jelly;
the second freezing step is carried out at the final freezing temperature, which is lower than the acclimation temperature;

### Lyophilization:

A primary lyophilization step is carried out through application of a vacuum of approximately 200 microbars and an ascending temperature profile;
a secondary lyophilization step is carried out through application of a vacuum of approximately 50 microbars and a descending temperature profile.

Following this lyophilization step, the product obtained is a disinfected, particularly virally inactivated and lyophilized, grind of Wharton's jelly. In addition, the product obtained is defined by a cylindrical shape with a diameter of 2 cm and a height of 0.3 cm, which is imparted by the support following the lyophilization step.

Each of the virally inactivated, and lyophilized grinds of Wharton's jelly formed in this manner is easily detached from its support and repositioned in the same support and placed in primary packaging, which is a TYVEK® sachet made of PE-PET copolymer.

A final step of sterilization of the ground, virally inactivated, and lyophilized Wharton's jelly is carried out by exposing this ground material to gamma radiation at 25-32 kGrays. All of the sachets comprising the ground materials obtained from the initial biological substance are treated simultaneously during this sterilization step by gamma radiation.

The grinds of virally inactivated and lyophilized and sterilized Wharton's jelly are recovered using a stainless steel spatula and fine curved stainless steel tweezers.

A virally inactivated, lyophilized, and sterilized biological material consisting of a disc of Wharton's jelly is obtained.

Alternatively, the biological material obtained is ground using a grinder, then passed through two successive sieves with pore diameters of less than 200 microns and 90 microns and subsequently packaged in glass bottles.

### Example 3: Preparation of a Lyophilized Biological Material According to the Invention Consisting of an Umbilical Cord Vessel

A properly informed and consenting donor in accordance with the requirements of the Declaration of Helsinki offers the umbilical cord resulting from childbirth as a donation. Due to health requirements relating to donations of tissues and cells of human origin, prior qualification of the donor is systematic. This qualification involves screening for HIV, hepatitis B, C, HTLV viruses, and the Treponema pallidum bacterium responsible for syphilis.

The umbilical cord is retrieved as soon as possible in the delivery room. It is advantageously placed in a sterile box comprising an NaCl solution at +4° C.

The following procedure is carried out in a sterile room in the laboratory: The segment that is inverted so that the Wharton's jelly is on the inside and mounted on the rigid guide is thawed in the open air, at room temperature, for a period of 5 minutes. The rigid PETG support is removed. This freezing step, followed by thawing, ensures the substantial devitalization of the biological material.

The inverted segment undergoes a succession of baths which ensure the chemical treatment thereof. This treatment aims to disinfect and, in particular, to virally inactivate the artery and Wharton's jelly. Gentle linear stirring of the liquid medium is applied during each bath to ensure homogeneous penetration of the solvents into the tissues.

First, the inverted segment is placed in a bath of purified water at room temperature for approximately 3 hours. This step ensures both the end of the thawing of the physiological tissue and a step of cell lysis by osmotic pressure.

The inverted segment is then transferred to a decontaminating bath composed of 70% v/v ethanol at room temperature for approximately 1 hour.

Washing is performed in purified water for approximately 15 minutes at room temperature in order to remove the ethanol.

To ensure the second step of decontamination treatment, the inverted segment is transferred to a bath composed of 30% w/v hydrogen peroxide at room temperature for approximately 15 minutes.

The inverted segment is then transferred to a decontaminating bath composed of 3% w/v hydrogen peroxide at room temperature for approximately 1 hour. The resulting segment is virally inactivated.

The chemical action applied to the virally inactivated segment is then neutralized in at least one bath comprising sodium hydroxide diluted to a pH of around 8.5. The treatment with at least one neutralization bath is carried out at room temperature for approximately 15 minutes.

In order to ensure a rebalancing of the pH and best eliminate the organic residues detaching from the tissue of interest, the virally inactivated segment is transferred to at least one bath of physiological buffer (PBS) in order to ensure the physiological rebalancing thereof. The at least one bath is carried out at room temperature for approximately 15 minutes.

Finally, the virally inactivated segment is transferred to two successive baths with purified water, at room temperature, for at least 15 minutes and up to approximately 1 hour.

At this stage of the method, we can consider that the segment obtained according to this chemical treatment is a largely disinfected, particularly virally inactivated, segment.

Following this stage relating to chemical treatments, the virally inactivated segment undergoes lyophilization.

A rigid sterile PETG support is inserted into the lumen of the virally inactivated segment. The latter is placed on a stainless steel tray.

This is all transferred to a lyophilizer, where a freezing step followed by a lyophilization step are carried out according to the following specifications:

### Freezing:

The first freezing step is carried out at an acclimation temperature that is selected so as not to damage the structural, functional, and biological integrity of the virally inactivated segment;
the second freezing step is carried out at the final freezing temperature, which is lower than the acclimation temperature;

### Lyophilization:

A primary lyophilization step is carried out through application of a vacuum of approximately 200 microbars and an ascending temperature profile;
a secondary lyophilization step is carried out through application of a vacuum of approximately 50 microbars and a descending temperature profile.

Following this lyophilization step, the inverted segment is a disinfected, particularly virally inactivated and lyophilized, segment.

A final sterilization step is carried out through exposure thereof to gamma radiation at 25-32 kGrays.

A virally inactivated, lyophilized, and sterilized biological material consisting of Wharton's jelly is obtained in the lumen of an umbilical cord vessel.

### Example 4: Method for Obtaining Exosomes from Mesenchymal Stem Cells

Mesenchymal stem cells from human umbilical cord are multiplied with fetal bovine serum to confluence.

Using a pipette, the media are collected in Amicon® Ultra 0.5 filtration tubes (Merk®) and are subjected to centrifugation at 4400 rpm for 30 minutes.

The exosome concentrate is collected in this manner.

The successful recovery of exosomes is validated using an ELISA test. A quantity of greater than 4.10⁹ exosomes is thus obtained.

### Example 5: Demonstration of the Release Capacity of a Biological Material Impregnated with a Solution Comprising Exosomes According to the Invention

A quantity of 150 μL of exosome concentrate obtained according to the protocol described in Example 4 is deposited on the surface of half a disc of Wharton's jelly obtained according to the protocol described in Example 2, with the other half-disc serving as a control.

After one minute, all of the exosome concentrate is impregnated into the half-disc.

The half-discs are then immersed in 1 mL of phosphate-buffered saline.

At the times 15 minutes, 30 minutes, 1 hour, and 4 hours after immersion, the medium is completely extracted for analysis and is replaced with a new milliliter of phosphate buffer saline.

The quantity of exosomes released into the medium is measured using an ExoELISA-ULTRA Complete Kit (CD63 Detection). The maximum dosage value using this kit is 4.06×10⁹.

| Sample | Quantity of exosomes |
|---|---|
| Half-disc according to Example 2 + Exosome | 4.06 × 10⁹ |
| according to Example 4 at T = 15 minutes | |
| Half-disc according to Example 2 + Exosome | 4.06 × 10⁹ |
| according to Example 4 at T = 30 minutes | |
| Half-disc according to Example 2 + Exosome | 3.99 × 10⁹ |
| according to Example 4 at T = 1 hour | |
| Half-disc according to Example 2 + Exosome | 3.90 × 10⁹ |
| according to Example 4 at T = 4 hours | |
| Control: half-disc according to Example 2 at | Below detection |
| T = 15 minutes | limit |
| Control: half-disc according to Example 2 at | Below detection |
| T = 30 minutes | limit |
| Control: half-disc according to Example 2 at | Below detection |
| T = 1 hour | limit |
| Control: half-disc according to Example 2 at | Below detection |
| T = 4 hours | limit |
| Solution comprising exosomes alone | >3.92 × 10⁹ |
| Solution comprising exosomes + 1 mL PBS | >4.00 × 10⁹ |

The biological material consisting of half a disc of Wharton's jelly impregnated with a solution comprising exosomes allowed for the sustained release of exosomes into the medium for 4 hours.

1-19. (canceled)

20. A biological material impregnated with a solution comprising exosomes, wherein the impregnated biological material originates from the impregnation of a lyophilized biological material with a solution comprising exosomes.

21. The impregnated biological material as set forth in claim 20, wherein the starting biological material comprises proteoglycans.

22. The impregnated biological material as set forth in claim 20, wherein the starting biological material is a connective tissue rich in proteoglycans.

23. The impregnated biological material as set forth in claim 20, wherein the starting biological material originates from one or more tissues of human origin.

24. The impregnated biological material as set forth in claim 20, wherein the starting biological material originates from placenta or umbilical cord.

25. The impregnated biological material as set forth in claim 20, wherein the starting biological material is Wharton's jelly.

26. The impregnated biological material as set forth in claim 20, wherein the starting biological material is an amniotic membrane.

27. The impregnated biological material as set forth in claim 20, wherein the impregnated biological material is virus-inactivated and/or sterile and/or lyophilized.

28. The impregnated biological material as set forth in claim 20, wherein the impregnated biological material comprises a therapeutically effective amount of exosomes according to the invention.

29. The impregnated biological material as set forth in claim 20, wherein the exosomes are derived from macrophages, blood platelets, dendritic cells, mesenchymal stem cells, induced pluripotent stem cells, bone marrow cells, adipose tissue, umbilical cord and/or are purified from biological fluids.

30. The impregnated biological material as set forth in claim 20, wherein the exosomes are derived from mesenchymal stem cells.

31. A biological material impregnation kit comprising at least two independent means:
a lyophilized biological material, and
a solution comprising exosomes.

32. The biological material impregnation kit comprising at least two independent means:
the lyophilized biological material, and
the solution comprising exosomes,
wherein the lyophilized biological material originates from a biological material as defined in claim 21.

33. The biological material impregnation kit comprising at least two independent means:
the lyophilized biological material, and
the solution comprising exosomes,
wherein the solution comprising exosomes is as defined in claim 27.

34. The impregnated biological material as set forth in claim 20 for use in therapy.

35. The impregnated biological material as set forth in claim 34 for therapeutic use in regenerative medicine and/or for the treatment and/or prevention of Crohn's disease and/or or fistulas and/or chronic bowel disease and/or graft-versus-host disease and/or intestinal inflammation and/or stroke and/or osteoarthritis and/or or respiratory distress syndrome and/or burns and/or cardiac myopathies and/or esophageal stenoses and/or chronic heart failure and/or cancer, particularly of the colon and/or breast and/or lung and/or pancreas and/or melanomas, and/or lysomal overload diseases, particularly Gaucher disease and/or Fabry disease, and/or mucopolysaccharidosis type III and/or Sanfilippo syndrome and/or bronchopulmonary dysplasia and/or chronic renal failure and/or mucositis after chemo- or radiotherapy treatment, and/or type 1 diabetes and/or gastroduodenal ulcers and/or pneumonia and/or venous ulcers and/or acute respiratory distress syndrome and/or Alzheimer's-related dementia and/or acute myocardial infarction and/or chronic post-surgical inflammation of the temporal bone and/or periodontitis and/or dry eye and/or macular degeneration and/or neuralgia and/or depression and/or dementia and/or dystrophic epidermolysis bullosa.

36. A method for obtaining an impregnated biological material, comprising the steps of:
providing a lyophilized biological material and a solution comprising exosomes,
bringing the lyophilized biological material into contact with the solution comprising exosomes, and
obtaining an impregnated biological material.

37. The method for obtaining an impregnated biological material comprising the steps of:
providing the lyophilized biological material and the solution comprising exosomes,
bringing the lyophilized biological material into contact with the solution comprising exosomes, and
obtaining the impregnated biological material,
wherein the lyophilized biological material originates from a starting biological material as defined in claim 21.

38. The method for obtaining an impregnated biological material comprising the steps of:
providing the lyophilized biological material and the solution comprising exosomes,
bringing the lyophilized biological material into contact with the solution comprising exosomes, and
obtaining the impregnated biological material,
wherein the exosomes are as defined in claim 20.
